(19) United States
(12) Patent Application Publication
(10) Pub. No.: US 2013/0184332 A1
(43) Pub. Date: Jul. 18, 2013

(54) Vectors Containing the Max Gene

(71) Applicant: Universidade Federal Do Rio De Janeiro, Rio de Janeiro (BR)

(72) Inventors: Rafael Linden, Rio de Janeiro (BR); Hilda Petrs Silva, Rio de Janeiro (BR); Luciana Barreto Chiarini, Rio de Janeiro (BR)

(73) Assignee: Universidade Federal Do Rio De Janeiro, Rio de Janeiro (BR)

(21) Appl. No.: 13/683,632

(22) Filed: Nov. 21, 2012

Related U.S. Application Data
(62) Division of application No. 12/936,352, filed on Dec. 29, 2010.

(30) Foreign Application Priority Data
Apr. 4, 2009 (BR) PI0800957-0

(51) Int. Cl. A61K 48/00

(52) U.S. Cl. CPC A61K 48/005
USPC 514/44 R; 435/455; 435/461; 435/459

(57) ABSTRACT

The present invention refers to the construction of cloning vectors containing the max gene. Especially, the present invention refers to the introduction of cloning vectors containing the max gene in cells using transport vectors. In addition, the presence of cloning vectors containing the max gene in cells allows the differential expression of the max gene in the same cells. In addition, the present invention refers to a method of gene therapy in which the differential expression of the max gene has cytoprotective activity, especially neuroprotective activity, and is capable of application to medical and veterinary therapeutics of neurodegenerative conditions.

### FIELD OF INVENTION

The present invention relates to the construction of cloning vectors containing the max gene. In particular, the present invention relates to the introduction of the cloning vectors containing the max gene in cells, using transport vectors. In addition, the presence of cloning vectors containing the max gene in cells allow for the differential expression of the max gene in those cells. In addition, the present invention refers to a method of gene therapy in which the differential expression of the max gene has cytoprotective activity, especially neuroprotective activity, which may be applied to medical and to veterinary therapeutics for neurodegenerative conditions.

### BACKGROUND OF THE INVENTION

### Gene Therapy

The explosive development of the recombinant DNA techniques and the sequencing of the human genome opened new perspectives for the management of both hereditary and acquired diseases of a genetic nature, as well as of other pathologies, including degenerative and infectious diseases, through gene therapy technologies.

The identification of genetic bases of the pathogenesis of certain diseases, some of which are due to mutation of a single gene, as well as of risk factors associated with or involved in either the pathogenesis or in the mechanisms of defense against disease, reveal opportunities for direct intervention upon gene expression with therapeutic purposes.

Gene therapy is the set of techniques to insert and express exogenous genetic material into cells with therapeutic purposes. The transfer of genetic material can be done in vivo (directly into the target organism) or ex vivo through the transduction of cells that are then inserted into the target organism. That is, gene therapy can be combined with cell therapies, inclusive of stem cells.

Conceived in the 1960s, gene therapy reached its first clinical trial by the end of the 1980s. The Journal of Gene Medicine records, by January 2009, a total of approximately 1,500 ongoing clinical trials. Among these, in all of Latin America, only one was recorded in Mexico, and none in South America. Research in gene therapy in Brazil has been boosted by the Gene Therapy Network, supported by the Ministry of Science and Technology and the National Research Council.

### Biological Vectors

Cells are normally resistant to the entry of exogenus DNA. To overcome this barrier, a vehicle is used for insertion of the gene (known as ‘transport vector’), to increase the probability of DNA entry. The most frequently used vectors in the current state of the art are derived from viruses, due to the fact that these microorganisms are naturally capable to infect living cells. However, it is also possible to use ‘non-viral’ vectors, such as plasmid DNA, to introduce DNA in exogenous cells. There are certain physical and biochemical techniques for the introduction of cloning vectors into cells. Among non-viral vectors, one can use microinjection, electroporation, plasmid injection, ballistic DNA injection, plasmids, liposomes, cationic compounds of lipid, protein or amidoprotein nature, such as DNA-calcium phosphate, DNA-DEAE dextran, DNA-lipid, DNA-lipid-protein and artificial chromosomes used in gene therapy. Among the viral transport vectors, one can use retrovirus, adenovirus, adenoassociated virus, hybrid adeno-adenoassociated virus, herpes simplex, lentivirus, foamy, HIV and vaccinia.

To use viral vectors for gene therapy, the viruses are modified by recombinant DNA techniques for the removal of all genes associated with viral pathogenesis, in order to obtain a minimal construction capable of infecting cells without viral replication and without adverse effects. A gene of interest can be added to this construct, and the resulting construct is used to insert the gene of interest in cells of the organism to be treated.

Nonetheless, the insertion and expression of a gene into an organism is an invasive process. Both the introduced gene as well as the viral vector may produce produce adverse effects related with the unbalance in gene expression, or the presence of non-self antigens. The target organism reacts to the presence of a virus by activating inflammatory and immune responses, which may damage the target of the gene therapy. One of the most competitive areas of biotechnology applied to gene therapy is the search for vectors which are both safer and, at the same time, capable of productive infection of the target cells, as well as of maintaining long term expression of the gene of interest.

In particular, constructs derived from adeno-associated viruses (AAV) have been targeted as a potential vectors for gene transfer in human clinical trials. Among their most favorable properties are: (I) no link of adeno-associated viruses with human diseases; (II) ability to infect cell lines derived from distinct tissues; (III) persistence of AAV-derived vectors in episomal form, thus avoiding insertional mutagenesis; (IV) ability to infect non-dividing cells.

The capacity of transduction with recombinant AAV (rAAV) has been demonstrated in a wide variety of cell types, including differentiated cells, which suggests a great potential of this vector system for in vivo gene transfer to organs such as muscle, liver, central nervous system and lungs. The rAAV vectors are derived from plasmids that carry the inverted terminal repeats (ITRs) flanking the exogenous gene of interest. The inverted terminal repeats are symmetrical sequences of 145 bases each, which are required for replication of the AAV DNA. Traditionally, rAAV vectors used to be produced by co-transfection of HEK293 cells with 2 plasmids, one containing the gene of interest flanked by ITRs, and a second plasmid containing the packing genes rep and cap, together with a helper virus (adenovirus or herpesvirus), the role of which was to provide ancillary factors for the replication of AAV. Modern techniques, however, employ only the co-transfection of a main AAV plasmid containing the gene of interest and its regulatory sequences flanked by ITRs, plus a helper plasmid containing all the minimal sequences required for packaging. This procedure, besides increasing production yield, aims at avoiding the contamination of rAAV preparation by adenovirus, which are pathogenic and, for this reason, were replaced by the minimal helper plasmid. The rAAV are recovered after cell lysis, and the helper plasmid is removed together with the DNA of the packaging cell line. Thus, currently, three elements are required for packaging of the AAV: (I) eukaryotic cells in culture; (II) the main plasmid with the gene of interest and the replication sequence; (III) the helper plasmid with the viral replication genes.

Recombinant AAV vectors are, in general, non-pathogenic and are the safest among the currently available vectors. Therefore, expression platforms based on rAAV have a great potential for clinical use.

In the patent field, various documents describe the construction of transport vectors for gene therapy. Some examples follow:

U.S. Pat. No. 6,521,225 describes the construction of novel adeno-associated viral vectors, specifically for delivery of therapeutic molecules to the liver. This vector contains 2 inverted terminal repeats (ITRs), each one comprising a specific sequence of 5-15 nucleotides, allowing for one or more deletions or substitutions.

Another document, U.S. Pat. No. 6,482,634, describes methods for the construction of vectors useful for the production of recombinant AAVs. The described method includes a host cell containing one molecule of 5″-3′ nucleic acid, a P5 promoter from parvovirus, one spacer, one AAV rep sequence and one AAV cap sequence. The spacer reduces the expression of gene products rep78 and rep68. The second molecule of nucleic acid contains one minigene and this one, in turn, contains one transgene flanked by ITRs, which is under control of regulatory sequences directed to expression in the host cell and to essential functions for replication and packaging of rAAV.

Document WO0182973 describes both viral and non-viral vectors as vehicles for the delivery of transgenes for the treatment of bone pathologies, through the local administration of a vehicle of delivery comprising the transgene for patients without a compatible bone matrix. This document describes a delivery viral or non-viral vector comprising genetic information related to therapeutic osteoinducer factors for target cells in vivo, forcing the cells to produce osteoinducer factors at the location of the bone pathology.

These documents do not overlap the present invention, because the biological vectors in question do not comprise the cloning vector containing the max gene, nor cite its use in neuroprotective therapies.

### Neurodegenerative Diseases

There are several recent reviews in the literature concerning applications of gene therapy in the areas of neurodegenarative diseases. In one particular case, the retina, which is a part of the central nervous system, is subject to neurodegenerative diseases known as degenerative retinopathies or retinal dystrophies, which are current targets for the development of gene therapy.

Examples of the combination of an adequate gene to a certain neurodegenerative pathology with a safe viral vector, in some cases already in clinical trials, can be found for the treatment of Parkinson's disease, Leber's congenital amaurosis and Alzheimer's disease.

Various patent documents describe gene therapies for neurodegenerative diseases. An example is document U.S. Pat. No. 6,683,058, which describes methods for the treatment of cerebral neurodegenerative diseases.

This document describes a form of therapeutical delivery of neurotrophins to a target (damaged, diseased or dysfunctional cholinergic neurons). The latter receive a transgene coding a neurotrophin within the mammalian brain, with the ensuing delivery of a defined concentration of recombinant neurotrophins by long periods.

This document differs from the current invention by describing a biological vector with therapeutic properties for cerebral diseases involving neurotrophins, with no relationship with any modification of the expression of the max gene with the use of that vector.

### Programmed Cell Death

The treatment of degenerative diseases in general, and neurodegenerative diseases in particular, is often frustrated by the multiplicity of factors that affect the sensitivity of cells to programmed cell death (PCD). In these cases, there is usually no single therapeutic target sensitive to drugs and, therefore, the usual therapies are very limited, with frequently non significant results.

Currently there is no cure or even long term effective treatments for most neurodegenerative diseases. This class of disease is characterized by increased rates of PCD among cells of the nervous system, particularly neurons. Their pathogenic mechanisms may include mutations, as in Huntington's diseases, changes in the supplies of oxygen and nutrients, such as in cerebrovascular diseases, or a complex balance of multiple genetic and epigenetic components, such as in Alzheimers's disease. Research in this field is vigorous worldwide, and concentrated, on the one hand, in the study of the mechanisms of cell death and cytoprotection, and, on the other hand, in the design of experimental strategies for recovery or functional preservation of organs or tissues, aiming at reducing cellular stress and the probability of PCD.

In the patent field, the use of viral vectors for modulating PCD may be illustrated by document U.S. Pat. No. 7,256,181. Therein, a method for the treatment of cancer is described, consisting of parenteral administration of interferon-.beta. through a viral vector for in vivo gene therapy. It is know that interferon-.beta. is part of a pathway of induction of the p53 gene, which codes for a molecule that can induce apoptosis in face of large scale cellular damage.

That document does not overlap the current invention, because it amounts to gene therapy for the induction, and not blockade, of PCD, refers to various viral vectors and uses the gene that codes for interferon-ft with no relationship with the max gene or other transcription factors.

Another example, this time related to the potential use of viral vectors acting upon pathways of PCD, is found in document U.S. Pat. No. 6,998,118. This document describes methods for neuronal transduction using rAAV vector injected around the synaptic portion and transported along the axons of the neurons towards their cell bodies, thus allowing the expression of unspecified genes. In the described method, adeno-associated viral vectors are used that can be transported through retrograde axonal transport, to introduce or express genes in neurons, and can also be applied to the tracing of neural pathways, in the stimulation or inhibition of neuronal growth and in the treatment of diseases such as Alzheimer's, depending on the gene inserted in the vector.

Since that document describes generically the use of gene therapy to allow the entry and expression of genes in neurons without specification of the genes of interest that may be applied to each case, it does not overlap the current invention, which describes specifically a vector for gene therapy to control neurodegeneration through the control of the expression of the max gene.

### Neuroprotective Therapies

The most promising alternative to face the multiple factors involved in the course of neurodegenerative diseases is the development of neuroprotective therapies, that is, those that diminish in general the sensitivity of cells of the nervous system to PCD. Pharmacological methods based either on neurotrophic factors (a class of growth factors with action upon the nervous system) or on other neuroprotective molecules have been shown to be of little efficacy, because of the need of repetitive administration of the drugs. This means either repetitive injections or continuous infusion directly into the nervous tissue or in the cerebrospinal fluid to overcome the blood-brain barrier, which blocks either partially or completely the distribution of drugs from the blood to the nervous tissue. The solution to avoid the risk associated with repetitive manipulation of the cerebral tissue lies upon the development of therapies based on a reduced number of interventions, ideally a single one, with permanent results.

Such an objective can be achieved through the development of gene therapy based on the discovery of target genes capable to protect the nervous tissue against multiple factors acting upon PCD. However, the identification of target genes for gene therapy of neurodegenerations is not trivial. Even in cases such as Huntington's disease, there is no single gene amenable to direct approach (which is usually the case in monogenic diseases). In the few preliminary gene therapy assays so far, that appear to block the clinical deterioration of patients of neurodegeneration, the procedure is specific for that condition, on the basis of selective sensitivity of a neuronal population to the presence of a certain neurotrophic factor, or to the expression of a certain gene for the functional recovery of a specific neuronal circuit within the central nervous system.

The target of the gene therapy could possibly be the whole of the mechanisms of PCD. Several forms of execution of PCD have been identified, triggered as a consequence of multiple types of cellular stress. The best known form of execution is PCD by apoptosis. Nevertheless, the experimental studies have clearly shown various pathways of apopotosis, as well as other alternative forms of PCD. It is also known that the blockade of certain pathways of execution does not prevent cells from dying by alternative routes, which may be activated, for example, upon blockade of apoptosis itself [Guimaraes C A and Linden R—Programmed cell deaths. Apoptosis and alternative deathstyles. Eur J. Biochem. 2004 May; 271(9):1638-50].

Certain experimental gene therapy procedures developed so far are based on either the introduction or the differential expression (overexpression) of genes, which, on the one hand, have a cytoprotective effect, but on the other hand are potentially capable of producing cancer. This conflict occurs because cancer is, essentially, the result of the loss of balance between mechanisms of cell proliferation and PCD, in which the inhibition of the latter has oncogenic potential. Therefore, an additional challenge to gene therapy for neurodegenerative diseases is to develop methods of neuroprotection based on the expression of a cytoprotective gene without oncogenic potential.

In the patent field, the importance of maintaining the balance cytoprotection/oncogenesis may be found in document U.S. Pat. No. 7,186,699, which describes an adeno-associated viral vector coding VEGF-TRAP, which is used to deliver and express genes coding for one or more anti-angiogenic of PCD-inducing genes, with an anti-tumoral effect, either associated or not with chemo- or radio-therapeutics.

This document does not overlap the current invention, because it does not refer to the expression of neuroprotective factors. On the contrary, that patent relates to methods of induction of PCD, whereas the present document relates to the prevention of PCD.

### Retinal Ganglion Cells and the Max Gene

Among the various populations of cells of the vertebrate retina, the retinal ganglion cells (RGC) are the neurons whose axons form the optic nerve and are responsible for the transmission of the information processed within the retina to the upper levels of the visual system in the brain. Their degeneration results in blindness.

In general, the methods of therapeutic intervention against PCD developed so far act upon relatively late stages of the process of cell degeneration, when the mechanisms of execution of PCD are already irreversible, without perspective of recovery or functional maintenance. Therefore, novel solution for intervention upon mechanisms of PCD must be directed at the earliest possible stages of transition from cell stress to the triggering of cell death execution mechanisms.

Experimental studies of the mechanisms of PCD in the rodent retina have shown that retinal neurons and progenitor cells present nuclear exclusion of transcription factors, that is, transcription factors that are normally found in the nucleus of healthy neurons appear in the cytoplasm of these cells when the latter are subject to distinct forms of cellular stress or lesions [Linden R, Chiarini L B. Nuclear exclusion of transcription factors associated with apoptosis in developing nervous tissue. Braz J Med Biol Res. 1999 July; 32(7):813-20].

A subsequent experimental study has shown that the transcription factor Max is excluded from the nucleus of the RGC early upon transection of their axons, and independent of the activity of caspases (proteases involved in neuronal death by apoptosis) [Petrs-Silva H, de Freitas F G, Linden R, Chiarini L B. Early nuclear exclusion of the transcription factor max is associated with retinal ganglion cell death independent of caspase activity [J Cell Physiol. 2004 February; 198(2):179-87]. Another study from the same group examined mechanisms of nuclear exclusion of Max and discovered that the loss of this protein from the nucleus is due to degradation by the ubiquitin-proteasome system inside the nucleus, together with retention of newly-synthesized Max in the cytoplasm [Petrs-Silva H, Chiarini L B & Linden R—Exp Neurol. 2008 September; 213(1):202-9]. All these events occur in early stages, before the commitment of RGC to the mechanisms of execution of cell death.

The max gene codes for two isoforms of 21-22 kDa of the Max protein. Max functions in the control of the transcription of genes dependent of the activity of the transcription factor coded by the oncogene c-Myc. To function as a transcription factor, c-Myc must heterodimerize with Max. In turn, Max is capable of heterodimerization both with c-Myc, and with other proteins that comprise a network of transcription factors, such as Mnt and Mxd1-4 (previously known as Mad1, Mxi1, Mad3 and Mad4), as well as of homodimerization. The Myc-Max heterodimers and the Max-Max homodimers have affinity for the same elements in genomic DNA, and have opposed effects. Thus, the Max-Max homodimers antagonize the oncogenic effects of the Myc-Max heterodimers, and therefore, Max has an anti-oncogenic effect upon cells transduced with the max gene.

In the patent field, certain documents describe the gene max related with viral vectors. U.S. Pat. No. 5,693,487 describes nucleotide sequences coding the max gene, that result in a helix-loop-helix protein which forms a complex when binding to either Myc or Mad, which is capable of binding to DNA in a sequence-specific way. The document refers to molecules of nucleic acid which are capable of hybridization under certain conditions to nucleotide sequences of max cDNAs, or to nucleotide sequences of mad cDNAs. The Max polypeptide, when associated with polypeptides Myc or Mad is capable of binding to nucleotide sequences containing CACGTG.

U.S. Pat. No. 5,302,519 describes a nucleotide sequence which codes for the polypeptide Mad, capable of binding to the polypeptide Max and to inhibit the binding of Max to the nucleotide sequence CACGTG.

None of the described documents overlap the present invention, because they relate to the specific interaction of nucleotide sequences of gene max with other sequences, and relate to the expression of the polypeptide Max exclusively with respect to binding to a specific DNA sequence.

U.S. Pat. No. 5,512,473 refers to the nucleotide sequence and expression of the polypeptide Mxi1, which interacts with polypeptide Max. This document does not overlap the present invention, because it refers to another DNA sequence and another polypeptide that interacts with Max.

In turn, documents U.S. Pat. No. 5,811,298 and U.S. Pat. No. 6,140,476 describe plasmidial or viral vectors containing a chimeric gene composed by the coding sequence for a repressor domain of gene Mxi, fused to the sequence that codes for gene Max. The chimeric gene expressed by these vectors codes for a fusion protein called Rep-Max, which specifically blocks the tumor promoting activity of oncogenic proteins of the c-Myc family. The cited documents do not overlap the present invention because: a) they refer to a fusion Mxi-Max construct, including only the bHLH, LZ and carboxy-terminal Max domains, that is, without the 5′ORF domain of gene max which codes for the N-terminal Max domain that contains, for example, the phosphorylation sites of the Max protein, that is, a construction that is fundamentally distinct from the Max construction employed in the present invention; (b) relate to the repressor effect of the Mxi polypeptide domain. instead of the effect of the polypeptide Max described in the present invention; (c) relate specifically to the repressor effect of the Mxi domain upon the tumor promoting activity of c-Myc family oncoproteins, distinct from the neuroprotective effect of Max described in the present invention; (d) relate to the effect of the Max polypeptide exclusively as a facilitator of the repressor effect of the Mxi domain of the fusion polypeptide, distinct from the direct effect of the Max polypeptide described in the present invention; (e) relate to the anti-oncogenic activity of the vectors containing the chimeric construction rep-max, distinct from the neuroprotective activity of the vectors containing max described in the present invention.

The described documents do not overlap the present invention because they relate to a chimera employing the max gene, and not the vector containing the max per se. In addition, the referred document does not cite the addition of the max gene to viral vectors with the aim of modulating the expression of the max gene with neuroprotective activity.

No documents were found citing cloning vectors containing the max gene, transport vectors containing the max gene, nor their use in cytoprotective, especially neuroprotective therapy.

The present invention relates to constructs of cloning vectors containing the max gene. In particular, the present invention refers to the introduction of cloning vectors containing the max gene in cell using transport vectors. In addition, the presence of the cloning vectors containing the max gene in cells allows the differential expression of the max gene with cytoprotective activity, especially neuroprotective activity, and may be applied to medical and veterinary therapeutics in neurodegenerative conditions.

### OBJECT OF THE INVENTION

In one aspect, this invention consists of biological vectors containing the max gene, comprising:
a) a cloning vector, comprising:
   a.1) a nucleotide sequence capable of expressing the max gene or its fragments;
   a.2) an adequate promoter;
b) transport vectors, chosen among the group that comprises viral transport vectors, non-viral transport vectors, and mixtures of both.

In certain embodiments, the cloning vector of a) comprises vectors of the group of plasmids, viruses, cosmids, and/or YACs.

In certain embodiments, the promoter of b) is any sequence capable of directing the expression of the max gene and/or its fragments in a cell.

In certain embodiments, the promotor is preferentially chosen among the group that comprises promoters CBA, CMV and/or hybrids CBA/CMV and/or specific promoters for each one of the various cell types.

In certain embodiments, the promoter is chosen among the groups of neuron-specific promoters.

In certain embodiments, the non-viral transport vectors are chosen among the group that comprises plasmids, liposomes, cationic lipid compounds, DNA-DEAF dextran, DNA-lipid, DNA-protein, DNA-lipid-protein, artificial chromosomes, nanoparticles, microinjection, eletroporation, injection of plasmid, ballistic injection of DNA.

In certain embodiments, the viral transport vectors are chosen among the group comprising adenovirus, adeno-associated virus, recombinant adeno-associated virus, retrovirus, herpesvirus, lentivirus, foamy, HIV, vaccinia.

An additional object of this invention consists of a method of production of biological vectors containing the max gene, comprising the stages of:
a) providing a cloning vector containing:
   a.1) a nucleotide sequence capable of expressing the max gene or its fragments;
   a.2) an adequate promoter;
b) providing a biological vector chosen among viral transport vectors, non-viral transport vectors, and mixtures of both; contacting the biological vector of b) with the cloning vector of a).

An additional object of the present invention consists of a method of expression of the max gene in cells, comprising the stage of contacting the biological vector with an adequate target cell, wherein the biological vector comprises:
a) a cloning vector comprising:
   a.1) a nucleotide sequence capable of expressing the max gene or its fragments;
   a.2) an adequate promoter;
b) transport vectors, chosen among the group that comprises viral transport vectors, non-viral transport vectors, and mixtures of both.

In certain embodiments, the biological vector is present in a pharmaceutically acceptable vehicle, chosen among the group that comprises pharmaceutically acceptable excipients and carriers, convenient doses and treatments for use in particular compositions that can be described in a series of treatment regimens, including oral, parenteral, intravenous, intramuscular, intracerebral, intracerebroventricular and intraocular.

In certain embodiments, the cell is a neuron of a higher animal, for example, human being.

An additional object of the present invention consists of a method of gene therapy comprising the stages of introduction of a biological vector containing the max gene in at least one target cell, where the differential expression of the max gene in the target cell will promote the modulation of cellular activity.

Preferentially, the modulation of cellular activity has a cytoprotective function.

Preferentially, the cell is a neuron of a higher animal, for example, human being.

### DESCRIPTION OF THE FIGURES

FIG. 1 shows a scheme of the transport vector pTR-CBA-max.

FIG. 2 shows a scheme of the transport vector pTR-SB-smCBA-max.

FIG. 3 shows a scheme of the transport vector pHpa-trs-SK-max.

FIG. 4 shows data on neuroprotection in vitro by rAAV-Max. Neonatal rats (on the first postnatal day) received injections of pTR-CBA-max in one eye and pTR-CBA-GFP in the other eye. After 14 days, the animals were terminally anaesthetized, their eyes were removed and their retinae were dissected. Explants of retinal tissue of approximately 1 mm.sup.2 were cut and placed in culture medium with 5% fetal calf serum for 30 hours, after which the tissue was fixed with 4% paraformaldehyde. The rate of cell death in the retinal ganglion cell layer was estimated by the percentage of pyknotic nuclei, indicative of cell death by apoptosis, with respect to the total number of cells in the sampling area (means and standard errors of the means in the histogram). The explants of the retinae transduced with rAAV-Max had approximately 50% lower cell death rates than the explants transduced with the control rAAV-GFP. Increased expression of Max was confirmed by immunohistochemistry, whereas the expression of the GFP was confirmed by the intrinsic fluorescence of the control protein.

FIG. 5 shows data on neuroprotection in vivo by rAAV-Max. Rats were distributed among 4 experimental groups as shown by the scheme. Two groups received injections of pTR-CBA-max in one eye, either on the first or on the 60th postnatal day. Three groups received lesions of the optic nerve on the 60th postnatal day (on the injected side, when injected) and all 4 groups were terminally anaesthetized and killed on the 74th postnatal day. Following fixation with 4% paraformaldehyde, the eyes were removed and the whole retinae were flat-mounted on gelatinized glass slides for adhesion of the fixed tissue. Nuclei of live cells or pyknotic profiles indicative of cell death were stained with the DNA intercalator sytox green, which labels DNA with green fluorescence, and were quantified in the ganglion cell layer by counting in photomicrographs obtained under laser confocal microscopy.

Counts were converted into nuclei (cell) densities and the results were presented in the form of means and standard errors of the means. The horizontal interrupted line indicates the mean density of displaced amacrine cells (in grey), found in the ganglion cell layer in the rat, which was discounted from the total to estimate the density of ganglion cells (in black). The retinae transduced with rAAV-max on the first postnatal day were protected against the death of the ganglion cells. The retinae transduced on the same day as the optic nerve lesion did not show significant protection, due to the time required for the expression of the transgene carried by the pTR-CBA-max vector (approximately 14 days).

FIG. 6 shows data from another example of neuroprotection in vivo by rAAV-Max. Rats were distributed in 4 experimental groups as shown in the scheme. All animals received, on the first postnatal day, multiple injections into the superior colliculus (projection target of the RGC axons) of a suspension of red fluorescent microspheres, which are transported along the axons to the cell bodies of the RGC. This procedure serves to positively identify this type of neuron, which constitutes a model of neurodegeneration in the central nervous system. Three groups received injections of pTR-CBA-max in one eye, on either the 30th or the 60th postnatal day. Three groups received optic nerve lesions by crush. on the 60th day (on the injected side, when injected), and all 4 groups were terminally anaesthetized and killed on the 74th postnatal day. Following fixation with 4% paraformaldehyde, the eyes were removed and the whole retinae were dissected and prepared as flat mounts on gelatinized glass slides for adhesion of the fixed tissue. The RGC were identified by the red labeling with the microspheres, whereas all nuclei of cells in the ganglion cell layer were labeled with the DNA intercalant sytox green (green). The RGC were quantified by counts in photomicrographs obtained by confocal microscopy. The counts were transformed in nulei (cell) densities and the results are shown in the form of means and standard errors of the means. Once again, transduction of RGC 30 days before the lesion produced neuroprotection (Petrs-Silva et al, unpublished results).

### DETAILED DESCRIPTION OF THE INVENTION

The examples below are not intended to restrict the scope of the invention, but rather to illustrate one of the many ways to implement the invention.

As used herein, ‘cloning vectors’ are vectors capable of amplifying the genetic information of a fragment of DNA inserted into an exogenous DNA and may be selected among a group that comprises plasmids, viruses, cosmids and/or YACs. The cloning vector contain a promoter, which is a sequence capable of promoting the expression of the DNA sequence in a cell. The promoter is preferentially chosen among a group that comprises CBA, CMV, and/or hybrids CBA/CMV, and/or specific promoters for target cells, such as for example, neuron-specific promoters.

As used herein, ‘transport vectors’ are the carriers of genetic information to target cells containing the cloning vector, and may be selected among a group that includes the viral and the non-viral transport vectors.

As used herein, ‘viral transport vectors’ are the group that comprises adenovirus, adeno-associated virus, retrovirus, herpesvirus, lentivirus, foamy, HIV, vaccinia.

As used herein, ‘non-viral transport vectors’ are those that comprise plasmids, liposomes, cationic lipid, protein and amidoprotein compounds, such as DNA-calcium phosphate, DNA-DEAE dextran, DNA-lipid, DNA-protein, DNA-lipid-protein, artificial chromosomes, nanoparticles, microinjection, eletroporation, injection of plasmid, ballistic injection of DNA.

As used herein, ‘biological vectors’ are cloning vector(s) inserted in at least one transport vector.

As used herein, ‘pharmaceutically acceptable’ is a formulation containing acceptable pharmaceutical excipients and carriers, well known by professionals in the field, as also is the development of doses and treatments amenable to use in particular compositions that can be described in a series of treatment regimens, including oral, parenteral, intravenous, intranasal, intravitreous, intramuscular, intracerebral, intracerebroventricular and intraocular, as well as its administration and/or formulation.

As used herein, ‘target cells’ are the cells that will benefit from the expression of the max gene. Such cells include cells of higher animals, such as for example, neurons.

As used herein, ‘modulation of activity’ is any modification in the expression of elements such as DNA, RNA and/or protein in the target cells, as well any change in the behavior of such cells during and/or after the expression of the max gene in the target cells, with the aim of promoting either the overexpression of the max gene or its silencing. In particular, in the present invention, the modulation shall be with the aim of promoting the overexpression of the max gene.

### Example 1

### Construction of Plasmids

The cDNAs for max21 and max22 were kindly provided by Robert N. Eisenman (Fred Hutchinson Cancer Research Center—Seattle, Wash.).

The rAAV main plasmids are constructed based on plasmid pTR-UF 11, kindly provided by William Hauswirth (University of Florida—Gainesville, Fla., USA). In these constructions, the transgenes flanked by AAV2 ITRs (inverted terminal repeats) have their expression controlled by a CBA (chicken .beta.-actin) promoter, which in turn is a hybrid of the immediate early enhancer of CMV (cytomegalovirus), with 381 base pairs, plus chicken-.beta.-actin-exon 1-intron 1 promoter, with 1352 bp. All this sequence is followed by an SV-40 polyadenylation signal.

The CBA-CMV combination produces great transduction efficiency in retinal cells, particularly in ganglion cells. However, the combination of promoter and enhancer can eventually, be modified for use in other cell types, especially other neuron types, within the scope of the present invention.

The used procedures apply, for example but not exclusively, to other plasmids such as: (a) pTR-SB-smCBA, which does not contain the coding sequence of the gene of bacterial resistance to neomycine, its eukaryotic promoter HSV-tk, nor enhancer PYF441 from polyoma virus, nor the polyadenylation signal of the bovine growth hormone, which turns the vectors derived from this plasmid capable of use in clinical assays in human patients; (b) pHpa-trs-SK, which is an rAAV vector containing a DNA double strand, distinct from the above mentioned vectors. The base vector pHpa was given to our group by Dr. William Hauswirth, from the University of Florida, who received it from Dr. J. Samulski (University of North Carolina, USA), and its construction aims at accelerating the expression of the transgene, by forming a vector containing a DNA double strand, thus skipping the relatively slow stage of synthesis of the complementary strand. In this case, the promoter is CMV (cytomegalovirus), but this can be replaced by other promoters, according to need and within the scope of the present invention. Experiments carried out in our laboratory showed that this vector expresses a transgene in the retina in up to 7 days, that is reducing by 50% the time needed for transgene expression (Petrs-Silva et al, unpublished results).

### Example 2

### Construction of Plasmids pTR-CBA-max21 and pTR-CBA-GFP

One pfu (1.5 U/50 .mu.l reaction) polymerase (Stratagene) was used for PCR (polimerase chain reaction) to generate the max clone flanked by the consensus sequence for cleavage by the NotI restriction enzyme. The primers used were forward (5′-gcggccgcatgagcgataacgat-3′) and reverse (5′-gcggccgcttagctggcctccat-3′). The generated clone was inserted into the TOPO bridge plasmid, following the protocol and reagents of the TOPO TA Cloning Kit (Invitrogen). The gene for GFP (green fluorescent protein) is routinely used as an experimental control. and had already been flanked by the NotI cleavage sequence in the pBIISK bridge plasmid. The plasmids containing inserts were digested with the NotI enzyme (Promega), in the concentration of 1 U/.mu.g DNA for 1 hour at 37.degree. C. The generated fragments were purified from agarose gels with the DNA cleaner kit (Invitrogen).

Once bearing the cohesive termination generated by NotI, the clones were ligated in the pTR-UF plasmid, also previously digested with NotI, dephosphorylated for 30 minute with SAP (shrimp alkaline phosphatase—Promega, 5 U/.mu.g DNA) at 37.degree. C., inactivated for 10 minutes at 75.degree. C., purified with phenol-chloroform (Sigma), and precipitated with 3.times. volumes of 80% ethanol and 10% volume of 3M sodium acetate. The ligation reaction was done overnight with 1 .mu.l of T4 DNA ligase (20,000 U/ml-Promega) in a final volume of reaction of 20 .mu.l, in an ice bath with temperature starting at 13.degree. C. and ending at 20.degree. C. Four .mu.l of the ligation product were used to transform 50 .mu.l recA.sup.- and recB.sup.-electrocompetent bacteria (SURE cells—Stratagene) by electroporation (25 .mu.FD, 200 Ohms and 1.25 Kvolts). The bacteria were then incubated for an additional 1 hour in LB medium (1% NaCl, 1% peptone, 0.5% yeast extract) without antibiotics, in an incubator shaker at 200 rpm, at 37.degree. C. and, then, were seeded in LB-agar plates (1% NaCl, 1% peptone, 0.5% yeast extract in 1.5% Agar) with added ampicillin (50 .mu.g/ml), in which they were grown for 14 hours at 37.degree. C., to generate isolated colonies.

Colonies selected by the antibiotic ampicillin were grown in 5 ml of LB medium with ampicillin for 14 hours in an incubator shaker at 200 rpm at 37.degree. C., to obtain enough plasmid for analysis. The bacteria in suspension were used to purify plasmids with the Wizard-plus mini-prep kit (Promega). The plasmids were analyzed for the presence and orientation of the clone, through DNA sequencing, using primers for the sequence of the pTR-UF plasmid: forward (5′-tctttttcctacagctcctgggcaa-3′) and reverse (5′-gcattctagttgtggtttgtccaaa-3′). The plasmids were also digested with the SmaI restriction enzyme (Promega), at a concentration of 1 U/.mu.g DNA at room temperature for 1 hour, to confirm the presence of the clone, its orientation and that of the ITRs, which, being repetitive sequences, are easily excluded from the plasmid by bacteria in long-term cultures.

### Example 3

### Production of Plasmids

For large scale transfection, which is needed for the efficient production of viral vectors, large amounts both of highly purified main plasmid as well as helper pDG plasmid are required. As mentioned above, the helper plasmid contains only minimal sequences required for packaging and avoids the presence of adenovirus as a helper virus for the packaging process.

### Example 3.1

### Maxi-Prep

Colonies of bacteria containing the constructs of interest were grown in suspension in 5 ml of LB medium with ampicillin, in an incubator shaker at 200 rpm 37.degree. C. for 5 hours, then transferred to 1 L of TB medium (1.2% Triptone, 2.4% yeast extract and 0.4% glicerol), with 10% KHPO buffer plus 0.1% ampicillin (100 mg/ml), pre-heated at 37.degree. C. The colonies in LB medium were left in the shaker at 37.degree. C. for another 14 h. To precipitate the bacteria, the medium was centrifuged for 10 minutes at 4000 rpm, using a JA-10 rotor. For each 250 ml of centrifuged medium, 20 ml of resuspension solution (50 mM Tris-HCl, pH8.0, 10 mM EDTA, pH 8.0, 20 .mu.g/ml RNAse) were added to the pellet, and the latter was dissociated with a pipet. Then, an additional 20 ml of a previously prepared lysis solution were added (0.2N NaOH, SDS 1% (w/v)). The mixture was shaken and incubated for 5 minutes at room temperature. Lastly, 20 ml of neutralization solution (3M potassium acetate, 28.7% acetic acid (w/v)) were added under shaking. The mixture was placed on ice for 10 minutes and then centrifuged for 10 minutes at 8000 rpm, using a JA-10 rotor. The supernatant was collected and 0.6 volumes of isopropanol were added, mixed and incubated on ice for at least 20 minutes. Then, the mixture was spinned for 20 minutes at 10.000 rpm, in a JA-10 rotor. The supernatant was discarded and the pellet was washed with 10 ml 80% ethanol, and left to dry at room temperature.

### Example 3.2

### Purification by Cesium Chloride Gradient

When dry, the pellet was carefully dissolved in 10 ml TE (100 mM Tris-HCl pH 7.6, 10 mM EDTA pH 8.0) with a pipette. Then, 1 g cesium chloride was added for each 1 ml of solution. The mixture was transferred to ultracentrifuge tubes (25.4.times.89.1 mm, Quick-Seal—Beckman), and 100 .mu.l ethidium bromide were added (740 .mu.g/ml). (Invitrogen). The tubes were sealed and centrifuged overnight, at 45,000 rpm, using a Vti-65 rotor. Following centrifugation, the lowest band in the tube stained with ethidium bromide was removed with a syringe and a #18 needle. The DNA was washed 3.times. with butanol saturated in H.sub.2O. Water was added at 2.5.times. the original volume of DNA, and ethanol was added at 2.times. of the total volume, the mixture was incubated on ice for 20 minutes and centrifuged for 20 minutes at 15,000 rpm, using the JA-10 rotor. The supernatant was discarded, the pellet was washed with 80% ethanol, dried at room temperature and resuspended in 1-2 ml H.sub.2O.

### Example 4

### Dosage of Plasmids

DNA concentration was measured by UV (ultra-violet light) absorption, using a spectrophotometer. Five .mu.l of DNA were added to 995 .mu.l H.sub.2O, and optical density was read at 260 nm excitation. To analyze the purity of the sample, a second measurement was made at 280 nm excitation. The ratio between the two readings at 260 nm and 280 nm was determined. When the ratio is between 2 and 1.5 the plasmids can be used for transfection. Below 1.5 there is the need for further purification.

### Example 5

### Cell Culture

A human embryonic kidney cell line (HEK-293) was maintained in complete DMEM (Dulbecco's modified Eagle's medium) (Gibco) supplemented with 10% fetal bovine serum (FBS—Gibco), 100 U/ml penicillin G (Gibco), and 100 mg/ml streptomycin (Gibco). The culture is kept in an incubator at 5% CO.sub.2, and 37.degree. C. The cells were passed from 1 to 3 culture flasks at every 3 days, using PBS for washing and 0.05% trypsin/0.53 mM EDTA in HBSS (Gibco) to dissociate the cells. One large scale transfection was done for the production of each vector. In each transfection, a cell factory (NUNC) of 6,320 cm.sup.2 with 10 shelves was used. One day before transfection, the cells were passed from 1 to 3 cell factories, and therefore, on the day of transfection, cells would be 75-80% confluent, adding to a total of 1.times.10.sup.9 cells.

### Example 6

### Transfection

Transfection was done by precipitation with calcium phosphate. A mixture was made of 1.8 mg of the helper plasmid pDG plus 0.6 mg of the main plasmid, such as to yield a 1:1 molar ratio of DNA, in 50 ml de CaCl.sub.2 0.25M, followed by addition of 50 ml 2.times.HBS, pH 7.05 (250 mM HEPES free acid, 1.4 M NaCl and 14 mM Na.sub.2HPO.sub.4). The 2.times.HBS solution is added last. The mixture was incubated for 1-2 minutes at room temperature and then added to 1100 ml complete medium. The cells' culture medium was removed and the new medium was added. The cells were incubated for 72 hours. At the end of this period, the culture medium was discarded, the cells were washed with PBS and dissociated in PBS containing 5 mM EDTA. The cells were then centrifuged at 1000 g for 10-15 minutes, the supernatant was discarded and the pellet was resuspended in 60 ml lysis buffer (150 mM NaCl, 50 mM Tris-HCl, pH8.4), and stocked at −20.degree. C. until the purification of the vectors.

### Example 7

### Purification of the Viral Vectors

### Example 7.1

### Cell Lysis and Iodixanol Gradient

Cells were lysed through 3 cycles of freezing on ethanol/dry ice, and thawing at 37.degree. C. Benzonase (Sigma) was added at a final concentration of 50 U/ml and incubated for 30 minutes at 37.degree. C. The lysed material was then centrifuged at 4000 g for 20 minutes and the supernatant containing the viral vectors was split in 4 gradients of iodixanol (5,5′-[(2-hydroxy-1,3-propanediyl)bis(acetyl-1,3-benzenecarboxamide] (OptiPrep—Axis-Shield). Iodixanol is a non-ionic medium for the formation of density gradients, which remains isosmotic at all densities, and increases the efficacy of purification of viral vectors, avoiding both the loss of infectivity and the toxicity of cesium chloride or sucrose (Zolotukhin, S. et al, 1999). The density of rAAV is about 1.266 g/ml, which is equivalent to 50% iodixanol. Therefore, a discontinuous gradient is generated in tubes (28.8.times.107.7 mm, Quick-seal—Beckman), by laying the 15 ml of the least dense cell lysate, followed by 5 ml 15% iodixanol (Sigma) in PBS-MK (PBS with 1 mM MgCl.sub.2 and 2.5 mM KCl), 15 ml 25% iodixanol, 15 ml 40% iodixanol, and finally 15 ml 60% iodixanol. The tubes were then sealed and centrifuged using a 70 Ti rotor, at 69,000 rpm (350,000 g) for 1 hour at 18.degree. C. Approximately 5 ml were aspirated with a #18 needle on a syringe from the interface between 40% and 60%. This material was stocked at 4.degree. C. or frozen for subsequent chromatography.

### Example 7.2

### Chromatography

The fraction obtain through the iodixanol gradient was then purified and concentrated by column chromatography. A 5 ml heparin HiTrap ionic exchange column (Pharmacia) was used in the FPLC AKTA system (Pharmacia), at 5 ml/minute and eluted with PBS-MK with 0.5M NaCl. The column was initially equilibrated at 5 ml/min with 25 ml buffer A (20 mM Tris-HCl, 15 mM NaCl, pH 8.5), then with 25 ml buffer B (20 mM Tris-HCl, 500 mM NaCl, pH 8.5), followed by 25 ml buffer A. The iodixanol vector fraction was diluted 1:1 in buffer A, and applied to the column, which was adjusted to a flow rate of 3-5 ml/min. After the sample had flown through the HiTrap column, it was washed with 50 ml buffer A. The vector was eluted in buffer B, fractioned in approximately 50 fractions of 1 ml, and the fractions containing vector were collected. The vector was then concentrated and desalinized in a Biomax 100K concentrate (Millipore) for 3 cycles of centrifugation in microfuge at maximum 14000 rpm speed for 1 minute. In each cycle, the virus was concentrated to 1 ml, followed by the addition of 10 ml de Ringer Lactate. The rAAV vectors were stocked at −80.degree. C.

### Example 8

### Analysis of the rAAV Proteins

### Example 8.1

### Extraction of Proteins

Fifteen .mu.l of each stock were mixed to sample buffer (45% glycerol 100%, 5% 2-.beta.-mercapto-ethanol, 2% SDS in Tris-HCl 0.5 M pH 6.8, and Bromophenol Blue), boiled for 5 minutes and applied to a 12% de polyacrilamide gel (Bio-Rad).

### Example 8.2

### Silver Staining

The vector stocks were examined for purity by analysis of the total proteins in polyacrilamide gels stained with silver, using a Bio-Rad kit.

### Example 9

### Titration

The stock of purified vector was treated with DNAse Ito digest any unpackaged contaminant DNA. Ten .mu.l of the purified vector stock were incubated with 10 U DNAse I (Boehringer) in 100 .mu.l of reaction mixture (50 mM Tris-HCl, pH 7.5, 10 mM MgCl.sub.2) for 1 hour at 37.degree. C. At the end of the reaction, 10 .mu.l of 10.times. proteinase K buffer (10 mM Tris-HCl, pH 8.0, 10 mM EDTA, 1% SDS) was added, followed by addition of 1 .mu.l Proteinase K (18.6 mg/ml, Boehringer). The mixture was incubated at 42.degree. C. for 1 hour. The viral DNA was purified in 2 extractions with phenol/chloroform, followed by one extraction with chloroform and precipitation with ethanol overnight at −20.degree. C., using 10 .mu.g glycogen as a carrier. The mixture was then centrifuged in a microfuge for 30 minutes, at maximum 14000 rpm speed. The precipitated DNA was resuspended in 100 .mu.l water. Each reaction mixture for PCR had 1 .mu.l viral DNA diluted in 2 series of dilutions of the standard plasmid, which can be the main plasmid used to produce the viral vector, with known concentration, but with a distinct gene from the vector-contained transgene. The best rates for this standard lie between 1 and 100 pg, and other dilutions used are equivalent to expression rates of 1, 10, 50 e 100 pg. Primers used refer to the sequence immediately up and downstream of the transgene insertion: forward (5′-agttattaatagtaatcaatta-3′) e reverse (5′-atcctttcagggtattccagta-3′). The products of each reaction were analyzed in 2% agarose (Invitrogen) gel, containing 0.5 .mu.g/ml ethidium bromide. The electrophoresis ran until the 2 bands were resolved such as to allow comparison. Images of the bands were acquired using an ImageStore 7500 UVP system (BioRad). The density of each band was measured using an image analysis software (ZERO-Dscan Image Analysis System, version1.0—Scanalytics). Rates were plotted as a function of the concentration of the standard DNA. The concentration of the viral vector stock corresponded to equal numbers of molecules of viral DNA and standard DNA.

### Example 10

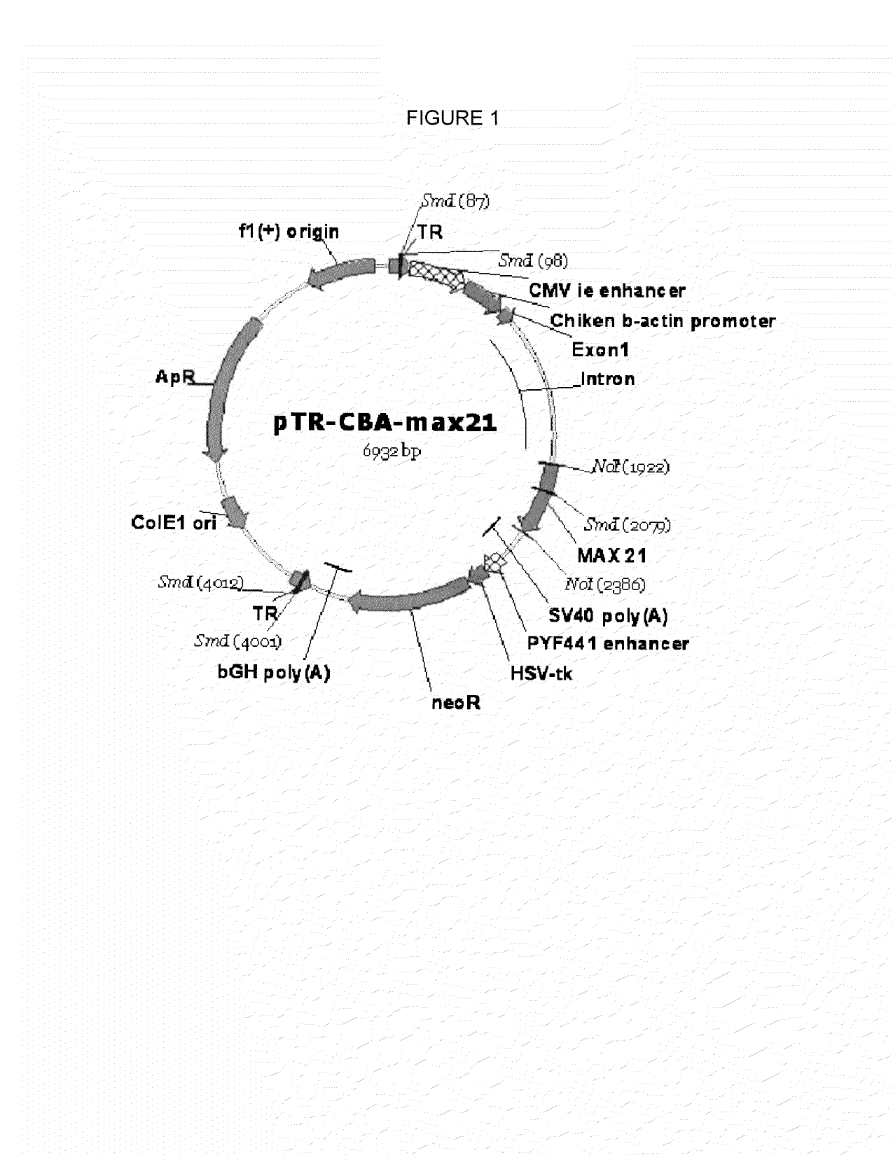

### Vetor pTR-CBA-max (FIG. 1)

Constructed upon a serotype 2 rAAV base vector (pTR), it contains a neomycin resistance gene for selection and a CBA (chicken beta-actin) general promoter, capable of promoting the expression of max in any cell type. Vectors derived from AAV show a relatively slow time course of expression, and reach maximum expression of the transgene about 14 days following transfection. The pTR-CBA-max vector reduced the sensitivity of retinal ganglion cells to cell death after transection of their axons in both in vitro and in vivo experiments.

### Example 10.1

### Sensitivity of Retinal Ganglion Cells to Cell Death After Transection of their Axons in Experiments In Vitro

Transduction of Max in Ganglion Cells: Neonatal rats (on the first postnatal day) received injections of the pTR-CBA-max in one eye, After 14 days, the animals were terminally anaesthetized, their eyes were removed and fixed by immersion in 4% paraformaldehyde in phosphate buffer. The eyes were cut in a cryostat and sections 10 micrometers thick were mounted in glass slides for immunohistochemistry. The Max protein was detected by reaction with a specific antibody, followed by development with a red fluorescent secondary antibody. The protein class III beta-tubulin, which labels RGC, was detected with a specific antibody, followed by development with a green fluorescent secondary antibody. Photomicrographs were obtained through laser confocal microscopy. Max protein content is clearly augmented in the RGC transduced with the pTR-CBA-max vector (mod. from Petrs-Silva et al, 2005).

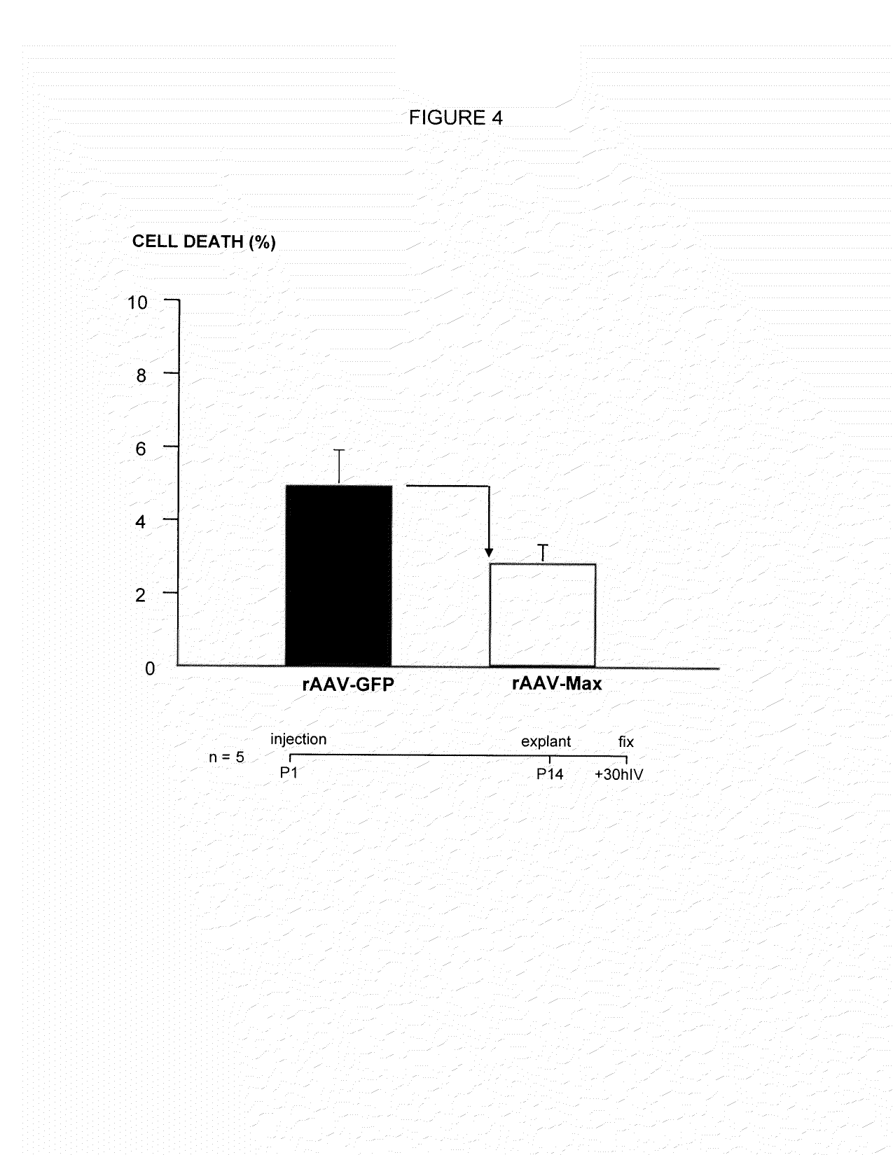

Neuroprotection In Vitro by rAAV-Max: Neonatal rats (on the first postnatal day) received injections of pTR-CBA-Max in one eye and pTR-CBA-GFP (control) on the other eye. After 14 days, the animals were terminally anaesthetized, their eyes were removed and the retinae were dissected. Retinal tissue explants of approximately 1 mm.sup.2 were cut and maintained in culture medium with 5% fetal calf serum for 30 hours, after which the tissue was fixed in 4% paraformaldehyde. The rate of cell death in the ganglion cell layer was estimated by the percentage of pyknotic profiles, indicative of cell death by apoptosis, with respect to the total number of cells in the sample (means and standard errors of the means in the histogram). The retinal transduced with rAAV-max had approximately 50% lower rates of cell death than the explants transduced with the control rAAV-GFP (FIG. 4). The increased expression of Max was confirmed by immunohistochemistry, while the expression of GFP was confirmed by the intrinsic fluorescence of the latter protein (Petrs-Silva et al, unpublished results)

### Example 10.2

### Sensitivity of Retinal Ganglion Cells to Cell Death Following Transection of their Axons in In Vivo Experiments

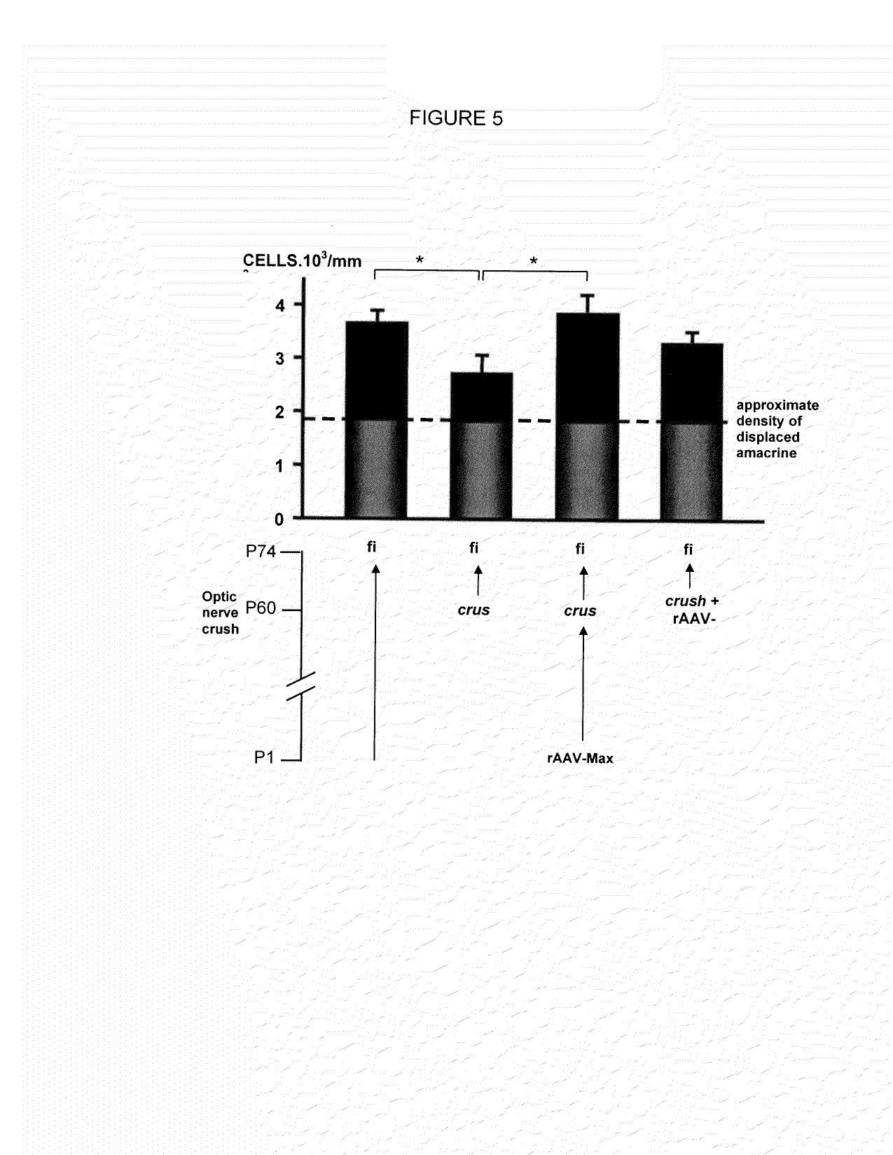

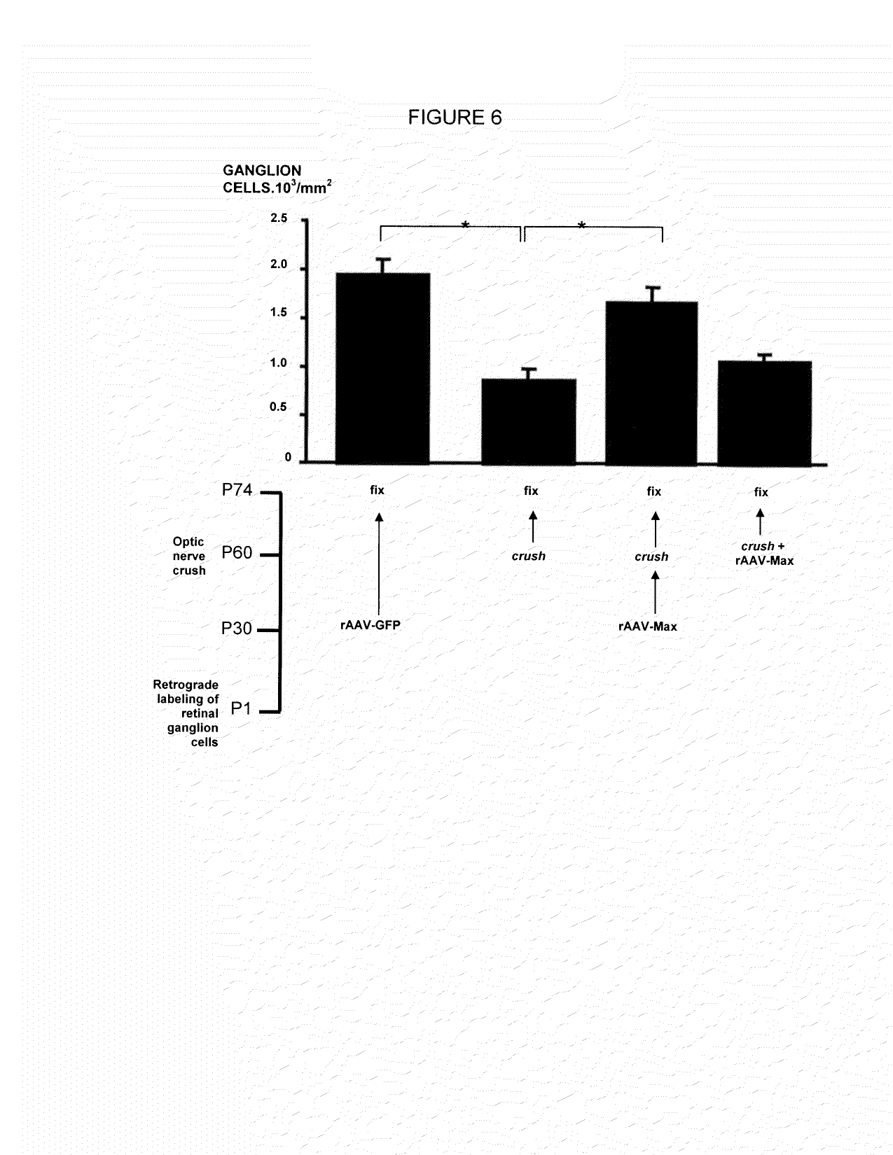

Neuroprotection In Vivo by rAAV-Max: Rats were distributed among 4 experimental groups as shown by the scheme of FIG. 5. Two groups received injections of pTR-CBA-max in one eye, either on the first or on the 60th postnatal day. Three groups received lesions of the optic nerve on the 60th postnatal day (on the injected side, when injected) and all 4 groups were terminally anaesthetized and killed on the 74th postnatal day. Following fixation with 4% paraformaldehyde, the eyes were removed and the whole retinae were flat-mounted on gelatinized glass slides for adhesion of the fixed tissue. Nuclei of live cells and pyknotic profiles indicative of cell death were stained with the DNA intercalator sytox green, which labels DNA with green fluorescence, and were quantified in the ganglion cell layer by counting in photomicrographs obtained by laser confocal microscopy. Counts were converted into nuclei (cell) densities and the results were presented in the form of means and standard errors of the means. The horizontal interrupted line indicates the mean density of displaced amacrine cells (in grey), found in the ganglion cell layer in the rat, which was discounted from the total to estimate the density of ganglion cells (in black). The retinae transduced with rAAV-max on the first postnatal day were protected against the death of the ganglion cells. The retinae transduced on the same day as the optic nerve lesion did not show significant protection, due to the time required for the expression of the transgene carried by the pTR-CBA-max vector (approximately 14 days) (Petrs-Silva et al, unpublished results).

Neuroprotection In Vivo by rAAV-Max: Rats were distributed in 4 experimental groups as shown in the scheme of FIG. 6. All animals received, on the first postnatal day, multiple injections into the superior colliculus (projection targets of the RGC axons) of a suspension of red fluorescent microspheres, which are transported along the axons to the cell bodies of the RGC. This procedure serves to positively identify this type of neuron, a models of neurodegeneration in the central nervous system. Three groups received injections of pTR-CBA-max in one eye, on either the 30th or the 60th postnatal day. Three groups received optic nerve lesions by crush. on the 60th day, on the injected side, when injected), and all 4 groups were terminally anaesthetized and killed on the 74th postnatal day. Following fixation with 4% paraformaldehyde, the eyes were removed and the whole retinae were dissected and prepared as flat mounts on gelatinized glass slides for adhesion of the fixed tissue. The RGC were identified by the red labeling with the microspheres, whereas all nuclei of cells in the ganglion cell layer were labeled with the DNA intercalant sytox green (green). The RGC were quantified by counts in photomicrographs obtained by confocal microscopy. The counts were transformed in nuclei (cell) densities and the results were shown in the form of means and standard errors of the means. Once again, transduction of RGC 30 days before the lesion produced neuroprotection (Petrs-Silva et al, unpublished results).

### Example 11

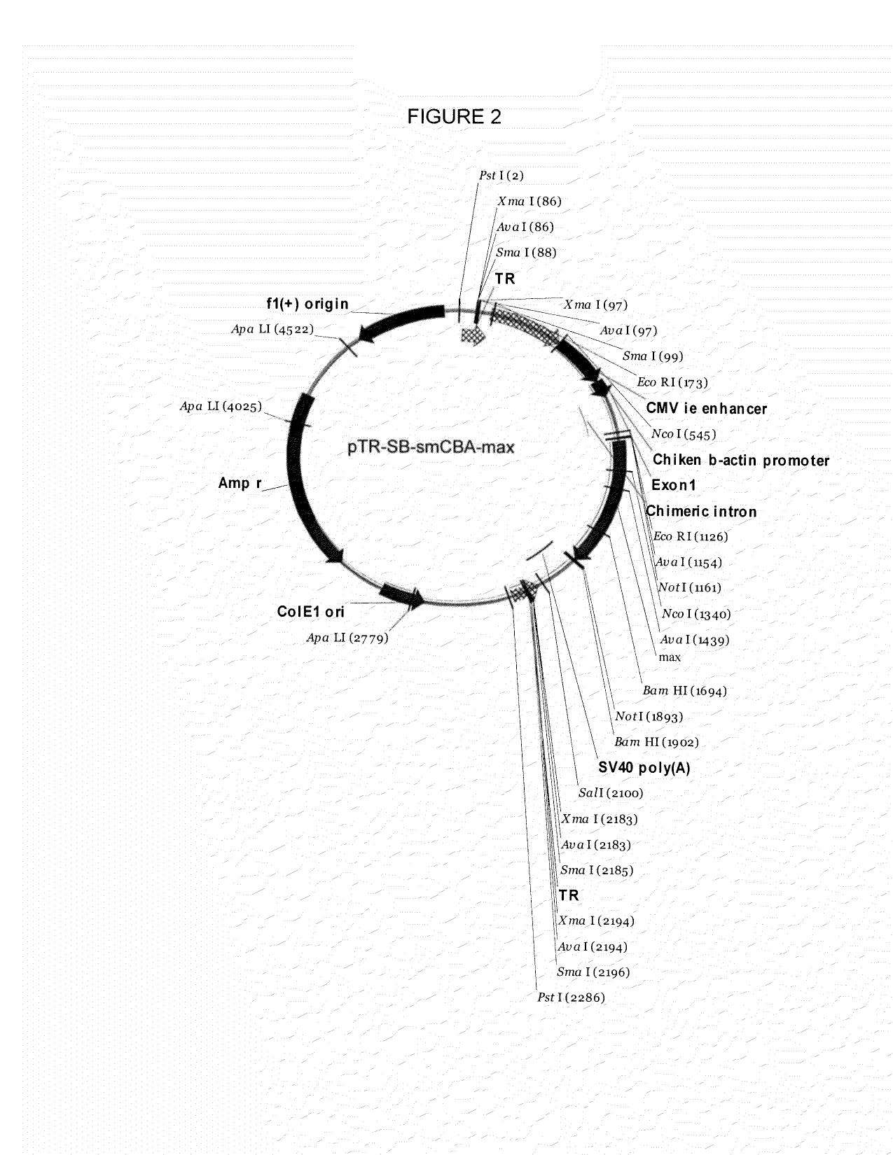

### Vector pTR-SB-smCBA-max (FIG. 2)

Under construction upon the serotype 2 rAAV base vector (pTR), similar to the base vector used in design 1. This vector had deleted the sequences coding for the neomycin bacterial resistance gene, its eukaryotic promoter HSV-tk, the PYF441 polyoma virus enhancer, and the bovine growth hormone polyadenylation signal. These modifications of not alter the speed of transgene expression, nor the distribution of the vector in the nervous system. The changes, however, make the vector amenable for use in human clinical assays.

### Example 12

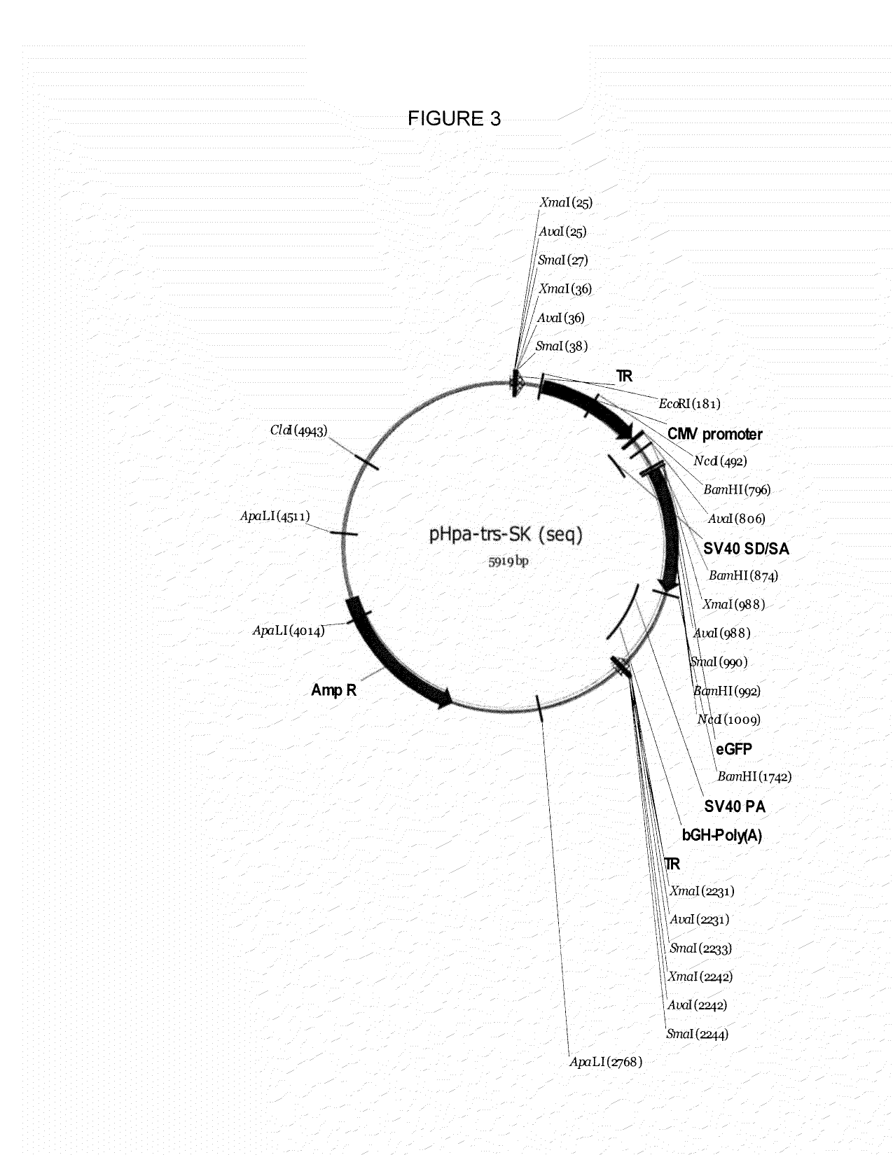

### Vector pHpa-trs-SK-max (FIG. 3)

Under construction upon a serotype 2 scAAV base vector (pHpa), distinct from the base vectors described above. The pHpa base vector was modified by the group of Dr. William Hauswirth, at the University of Florida, such as to accelerate the expression of the transgene due to the formation of a double strand, thus skipping the relatively slow stage of synthesis of the complementary strand. In this case, the promoter is CMV (cytomegalovirus). This type of vector expresses the transgene in up to 7 days, that is, reducing by 50% the time needed for transgene expression. Tests done in our laboratory confirmed the acceleration of the expression of the GFP transgene transduced by this vector in retinal tissue (Petrs-Silva et al, unpublished results).

1. A method for the expression of the max gene in cells, comprising the stage of contacting the biological vectors with an adequate cell, where the biological vector comprises:
a) a cloning vector containing:
a.1) a nucleotide sequence capable of expressing the max gene or its fragments; and
a.2) an adequate promoter; and
b) one or more transport vectors selected from a group consisting of viral transport vectors, non-viral transport vectors and mixtures of both.

2. The method for expression of claim 1, wherein the target cell comprises any cell of a higher animal.

3. The method for expression of claim 2, wherein the higher animal comprises a human being.

4. The method for expression of claim 2, wherein the target cell comprises a neuron.

5. The method for expression of claim 1, further comprising a cloning vector comprising vectors selected from a group consisting of plasmids, virus, cosmids and YACs.

6. The method for expression of claim 1, further comprising a nucleotide sequence chosen from a group consisting of sequence numbers 1, 2, 3, and 5, and mixtures thereof.

7. The method for expression of claim 1, further comprising a promoter chosen from a group consisting of promoters CBA, CMV and/or CBA/CMV and predetermined cell types.

8. The method for expression of claim 1, further comprising one or more non-viral transport vectors chosen from a group consisting of plasmids, liposomes, cationic lipid, protein and amidoprotein compounds, such as DNA-calcium phosphate, DNA-DEAE dextran, DNA-lipid, DNA-protein, DNA-lipid-protein, artificial chromosomes, nanoparticles, microinjection, eletroporation, injection of plasmid, ballistic injection of DNA, and mixtures thereof.

9. The method for expression of claim 1, further comprising one or more viral transport vectors chosen from a group consisting of adenovirus, adeno-associated virus, recombinant adeno-associated virus, retrovirus, herpesvirus, lentivirus, foamy, HIV, vaccinia, and mixtures thereof.

10. The method of expression of claim 1, further comprising a biological vector associated with a pharmaceutically acceptable vehicle.

11. The method of expression of claim 10, further comprising a pharmaceutically acceptable vehicle chosen from a group consisting of pharmaceutically acceptable excipients and carriers, convenient doses and treatments for use in particular compositions that can be described in a series of treatment regimens, including oral, parenteral, intravenous, intramuscular, intracerebral, intracerebroventricular and intraocular.

12. A method of gene therapy comprising a stage of introducing a biological vector containing a max gene into at least one target cell of a higher animal in order to modulate the cellular activity of the target cell.

13. The method of gene therapy of claim 12, wherein the higher animal comprises a human being.

14. The method of gene therapy of claim 12, the cell comprises a neuron.

15. The method of gene therapy of claim 12, wherein the modulation of activity of the target cell further comprises a cytoprotective function.

16. The method of gene therapy of claim 12, wherein modulation of activity of the target cell further comprises a neuroprotective function.


SEQUENCE LISTING (5 sequences)

SEQ ID NO: 1 - DNA, 2068 bp, Artificial Sequence
  MYC associated factor X (MAX), transcript variant 1 Oncoprotein
ggtggttcttgcccgtgttgtgtgtgtgtgtgagtgagagagcgagtgagtgagtgagtgagtgtgtgtgtgggggggactcggcttgttgttgtcggtgacttccccctccccttcaccccttcccctccccgccgccgctgcagtggccgctccctgggccgtaggaaatgagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagctaagccactcggggcaggccagcaataaaaactgtctgtctccatcgtctcatcctcctttcagttcgttggtagagccctcagaaccatttaagagactctttatttttctctttctcccttttttttttaaatttttatttttacgtagaagctcttggacaacagctctcgttctccttccccatttccactgtatattttttaatgtattcccttcagggattccctgtccccaacaggaatttttaaaccaaaacaccccaacttggcagctttttctgtggaggacagacggccggccggacctctgagcacatagtgtcctgcccaccctaccagctcctccagccctgccgggcacatgcccgggggacgcctgccctgcccaggtggcctcctggcccgccctcacctctgatagactttgtgaatctgaactgctctactttgagaagatgaccggtttggagtaatcagaatgaaccctcctcctttttaagggttttttttttttcctttttctaaaaagctatgtatcgctcctattgaaagaccagatccttagagaagtttgtggtataaaaaggaagtggggacagattcgcagcacagagtcgctggcatgtttcactcctgcttctctcagccagctgtttaagcctgcggcgccagcctcacggagggccgtgtgacactctcgtggtatgtatgggagatggcagcagtgaagcagcagccaccagggagtggccatttggggttgggacagggagggtgttttgggtggcatagaggttttgtattgagggccagtgatgatgttttgatatttatttcctgctacttaaatttgaatctgagtgaattgtacctatttctgatgatgtcggtcttgcaaagcgacagattcataaagtaatgatgaaatctttctttcttcccgtgtgtatttctaagaaatagagccaactgattttgtatgtaaataccaagagcaatttacctggtactaaacccgcaccccagtgcggacccttcccagccctcatcccacttcctttcctactgtcctggaacctgtctccattgtgtgatccagccctggttctggctgtggtcagcagatgccagtgaagggttttgtgtgtttaggcctcatttctttgtctttttcctactccgttcctggcatttgctgatttctagtgtatactctgtagtctcagttcgtgtttgattccattccatggaaataaaaagtatgttgtacatactgccgaagaattgtcttgcaagttaaggcttccccctttactataagactataaataaaaacttattttatccttaaaaaaaaaaaaaaaaaaaaaaaaaaaaaaaaaaaaaaaaaaaaaaaaaaaaaaaaaaaaaaaaaa

SEQ ID NO: 2 - DNA, 456 bp, Artificial Sequence
  MAX21 Oncoprotein
atgagcgataacgatgacatcgaggtggagagcgacgctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagctaa

SEQ ID NO: 3 - DNA, 483 bp, Artificial Sequence
  MAX22 Oncoprotein
atgagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagctaa

SEQ ID NO: 4 - DNA, 472 bp, Artificial Sequence
  Notl-MAX21-Notl Oncoprotein
gcggccgcatgagcgataacgatgacatcgaggtggagagcgacgctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagctaacgccggcg

SEQ ID NO: 5 - DNA, 499 bp, Artificial Sequence
  Notl-MAX22-Notl Oncoprotein
gcggccgcatgagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagctaacgccggcg